(19) United States
(12) Patent Application Publication
(10) Pub. No.: US 2025/0303016 A1
(43) Pub. Date: Oct. 2, 2025

(54) PLASMA SYSTEMS FOR AIR DECONTAMINATION AND AEROSOL ACTIVATION

(71) Applicant: The Trustees of Princeton University, Princeton, NJ (US)

(72) Inventors: Shurik YATOM, Hopewell, NJ (US); Sophia GERSHMAN, Scotch Plains, NJ (US); Yevgeny RAITSES,, Princeton, NJ (US); Philip EFTHIMION, Bedminster, NJ (US)

(73) Assignee: The Trustees of Princeton University, Princeton, NJ (US)

(21) Appl. No.: 18/872,939

(22) PCT Filed: Jun. 9, 2023
(86) PCT No.: PCT/US2023/024920

§ 371 Date: Dec. 9, 2024

Related U.S. Application Data
(60) Provisional application No. 63/350,539, filed on Jun. 9, 2022.

(51) Int. Cl. A61L 9/22; A61L 9/14

(52) U.S. Cl. CPC A61L 9/22; A61L 9/14; A61L 2209/14

(57) ABSTRACT

Disclosed are devices, systems, and techniques for decontaminating a gas or aerosol. Such techniques may be utilized, e.g., in conjunction with existing nebulizers or atomizers, or with residential, commercial, or industrial HVAC systems. The devices may include a housing configured to allow a gas or aerosol to pass from an inlet, through the housing, to an outlet. The devices may include a dielectric barrier discharge (DBD) element positioned in the opening or covering an end of the opening. The DBD element may be configured to generate a plasma and configured to allow the gas or aerosol to pass along or through a surface of the DBD element. When passing through the plasma, a disinfectant compound is created in the gas or aerosol, allowing for the gas or aerosol to be decontaminated. The decontaminated gas or aerosol can then be sent to, e.g., a mask, a room, etc.

### CROSS-REFERENCE TO RELATED APPLICATIONS

The present application claims priority to U.S. Provisional Patent Appl. No. 63/350,539, filed Jun. 9, 2022, the contents of which are incorporated by reference herein in its entirety.

### STATEMENT REGARDING FEDERALLY SPONSORED RESEARCH OR DEVELOPMENT

This invention was made with government support under Grant No. DE-AC02-09CH11466 awarded by the Department of Energy. The government has certain rights in the invention.

### TECHNICAL FIELD

The present application is drawn to the use of cold plasma for aerosol activation.

### BACKGROUND

This section is intended to introduce the reader to various aspects of art, which may be related to various aspects of the present invention that are described and/or claimed below. This discussion is believed to be helpful in providing the reader with background information to facilitate a better understanding of the various aspects of the present invention. Accordingly, it should be understood that these statements are to be read in this light, and not as admissions of prior art.

Plasma activated aerosols can be used for various treatments, but conventional techniques require use of noble gasses, such as Ar or He. The requirement of a noble gas supply introduces complications which limit the portability of, for example, an integrated plasma-activator plus nebulizer system, increases the system dimensions, and involves significant added costs.

### BRIEF SUMMARY

Various deficiencies in the prior art are addressed below by the disclosed devices, systems, and techniques.

In various aspects, a device may be provided. The device may include a housing. The housing may have walls defining an opening therethrough. The opening may be configured to allow a gas or aerosol to pass from an inlet, through the housing, to an outlet. The device may include a dielectric barrier discharge (DBD) element positioned in the opening or covering an end of the opening. The DBD element may be configured to generate a plasma. The DBD element may be configured to allow the gas or aerosol to pass along or through a surface of the DBD element.

The DBD element may include a conductive plate and a grounded metal mesh, separated by a thin dielectric film. The DBD element may include a plurality of conductive wires configured in a mesh or mesh-like pattern. The DBD element may include a plurality of electrodes, wherein at least one electrode comprises a plurality of small diameter high aspect ratio fibers. The DBD element may be coupled to at least a portion of an inner surface of the walls defining the opening.

In some embodiments, the gas or aerosol may be a gas (such as air, and may include oxygen). In some embodiments, the gas or aerosol may be an aerosol.

In various aspects, a system may be provided. The system may be a portable system. The system may be a non-portable system. The system may include a device as disclosed herein. The system may include a power supply (such as a battery) operably coupled to the device. The system may include an inlet channel configured to be operably connected to the inlet and provide a path directing at least one source of a gas or aerosol to the device. The system may include an outlet channel configured to be operably connected to the outlet and provide a path for a plasma-activated gas or aerosol to be transported away from the device.

The system may include a gas or aerosol filter positioned in the outlet channel or inlet channel. The system may include a fan positioned in the outlet channel. The system may include a heating and/or cooling element positioned in the outlet channel.

The inlet channel may be configured to receive return air from within an indoor space. The outlet chamber may be configured to return air to the indoor space. The inlet channel may be configured to receive return air from within an indoor space and fresh air from an outdoor space.

The inlet channel may be operably coupled to a source of a liquid. The system may include a nebulizer or atomizer configured to utilize the source of the liquid to generate an aerosol.

In various aspects, a method for air and aerosol decontamination may be provided. The method may include providing a device as disclosed herein. The method may include generating a plasma by causing a current to pass across the device. The method may include allowing a gas or an aerosol to pass into an inlet of the device, through the plasma, and out of an outlet.

In various aspects, a kit may be provided. The kit may include a device as disclosed herein. The kit may include a filter. The kit may include a fan. The kit may include a heating and/or cooling element. Alternatively, the kit may include a device as disclosed herein, a liquid source or reservoir for liquid; a nebulizer or atomizer, and a power supply.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration of a device.

FIGS. 2-4 are illustrations of cross-sections of DBD elements.

FIGS. 5 is an illustration of a system.

FIGS. 6-10 are illustrations of cross-sections of systems.

FIG. 11 is a block diagram of a system.

FIG. 12 is a cross-section of an axial view of a DBD element in a cylindrical arrangement.

FIG. 13 is a graph showing decontamination for different designs with different gas or aerosols over time.

FIG. 14 is a flowchart of a method.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

Disclosed herein are plasma systems for air decontamination and aerosol activation. The disclosed plasma system for aerosol activation easily integrates into a commercial, portable or desktop nebulizer and generates cold plasma in ambient air activating the mist flowing in the nebulizer tubing on its way to the mouthpiece. The disclosed device is a compact and lightweight add-on, easily mounted on nebulizer tubing. The disclosed approach for a plasma system for air decontamination includes several plasma source configurations that can be easily incorporated into air-flow ways in buildings that are routinely occupied by people, such as residential housing and workplaces. Cold plasma of two of the disclosed devices have been shown as a very effective approach for significant killing of bacteria and virus inactivation on surfaces, without inducing thermal or other damage. The disclosed approach may also be employed for air decontamination.

The disclosed plasma device for aerosol activation can be integrated in commercial nebulizers to enrich the water, solutions, and medications used in nebulizers with reactive species (RONS—Reactive Oxygen and/or Nitrogen Species).

The plasma activator device can be attached to a nebulizer and can generate plasma activated mist (PAMI). PAMI can be used for an effective reduction of bacteria and viruses in the upper respiratory tract. To that end, the oral cavity along with the throat are flooded with plasma-activated aerosol, by way of using the disclosed device. Due to its properties, the plasma-activated aerosol is distributed evenly and can reach not only the oral cavity and throat but also areas difficult to access, e.g., nasal passages. The plasma species will reduce the bacterial and viral load in the entire upper respiratory tract and can thus potentially prevent the spread of germs into the lungs.

PAMI has been shown to be effective in eradicating cancer cells and was disclosed as a post-surgical treatment to eliminate remaining cancer cells after surgery. Therefore, the disclosed device can be used for oncological therapy (see M. El Shaer et al., “Effect of Plasma Activated Mist on Breast Cancer Cells”, IEEE Trans. Rad. Plasma Med. Sci, 2, 103 (2018)).

PAMI has also showed significant beneficial impact on seed germination after application of PAMI to wheat seeds, therefore the disclosed device has potential application in agriculture (see M. El Shaer et al., “Germination of Wheat Seeds Exposed to Cold Atmospheric Plasma in Dry and Wet Plasma-Activated Water and Mist”, Plasma Medicine, 10, 1 (2020).).

The disclosed approach is a scalable device and can be added to, e.g., room-size nebulizers used to humidify or purify room air. PAMI can easily convert a device currently present in many homes into a personal disinfection and decontamination device particularly in situations requiring gentle action, such as when applied to skin and other sensitive surfaces. The antibacterial action of plasma activated media has been previously demonstrated.

PAMI generation has been investigated before in several works. In all cases, the plasma was created in a noble gas such as Ar or He. The mist from nebulizer or atomizer was then introduced into the plasma for activation. The disclosed device does not require noble gas flow to ignite the plasma, and therefore, is preferably free of a noble gas source. The plasma is ignited in the ambient atmosphere instead. The add-on device is small, light, and compact. It is powered by a small power supply (such as a supply that can provide AC voltages up to 100 kV and currents up to 0.3 mA), which may also be light and compact and can be integrated in the nebulizer packaging.

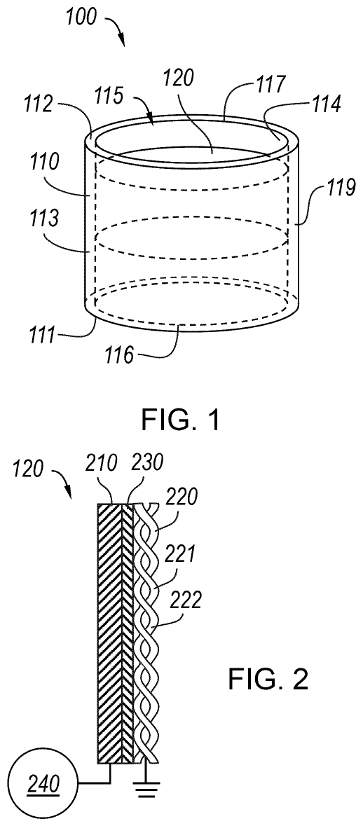

In various aspects, a device may be provided. Referring to FIG. 1, a device 100 may include a housing 110. The housing may have walls 119, which may have an outer surface 113 and an inner surface 114. The walls (which may be, e.g., sidewalls) may define an opening 115 extending through the housing. In some embodiments, the housing may be a tube or pipe.

The opening may form an inlet 116 at a first end 111 and an outlet 117 at a second end 112 opposite the first end. In this configuration, a gas or aerosol may be able to pass from the inlet, through the housing, to the outlet.

The device may include a dielectric barrier discharge (DBD) element 120 positioned in (or disposed within) the opening 115. In some embodiments, the DBD element is disposed at first end of the housing. In some embodiments, the DBD element is disposed at the second end of the housing. In some embodiments, the DBD element may be disposed at an intermediate location, between at a distance >0 from the first end and a distance >0 from the second end.

The DBD element may be coupled to at least a portion of an inner surface 114 of the walls defining the opening.

The DBD element may be configured to generate a plasma. The DBD element may be configured to allow the gas or aerosol to pass along or through a surface of the DBD element.

Referring to FIG. 2, the DBD element may include a conductive plate 210 and a grounded metal mesh 220, separated by a thin dielectric film 230. In some embodiments, the dielectric film may be no more than 5 mm thick. In some embodiments, the dielectric film may be no more than 4 mm thick. In some embodiments, the dielectric film may be no more than 2 mm thick. In some embodiments, the dielectric film may be no more than 3 mm thick. In some embodiments, the dielectric film is no more than 1 mm thick. In some embodiments, the dielectric film may be a coating around the metal forming the metal mesh.

The conductive plate may be comprised of a metal (such as copper). The conductive plate may be comprised of a polymer. The conductive plate may be operably coupled to a power source 240.

The DBD element may include a plurality of conductive wires 221, 222 configured in a mesh or mesh-like pattern.

In some embodiments, the element may consist of powered conductive plate (made of, e.g., a flexible copper tape) and a grounded, flexible metal mesh, separated by a thin, flexible, dielectric layer or film (e.g., polyamide). The device may be powered by an AC power supply. The device may be manufactured in a planar configuration, and reconfigured to, e.g., a cylindrical configuration. Such a configuration may be best suited for, e.g., an air decontamination application.

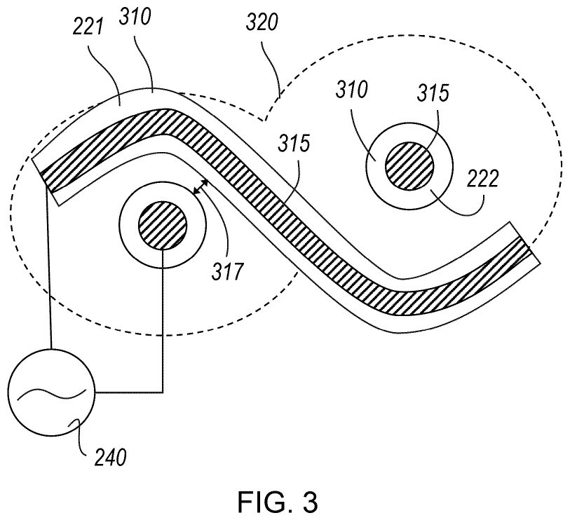

Referring to FIG. 3, the DBD element may include two or more wires 221 oriented in substantially a first direction, and a plurality of wires 222 oriented in a second, different, direction. Each wire may include a conductive core 315 (such as a metal, such as copper, or a conductive polymer), each of which may be surrounded by one or more non-conductive coatings 310 (such as a dielectric coating). The coating may be, e.g., polytetrafluoroethylene (PTFE) and expanded polytetrafluoroethylene (ePTFE) and related materials, polysiloxane or other silicon-based polymer materials, or a combination thereof. When current (e.g., from a power supply 240) is applied across the conductive cores, a plasma 320 may be created in a gas or aerosol (such as air recirculating in a building, or mist from a nebulizer) around the locations where a distance 317 between the wires is zero or relatively small (e.g., 2 mm or less).

In a preferred embodiment, the DBD element may include two wires. For example, an embodiment referred to as a plasma “weave” consists of two conducting fibers. A pulsed AC voltage is applied between these two wires. One or both of the wires may be covered in a form of insulation (e.g., a dielectric coating). A dielectric barrier discharge forms along the dielectric surface.

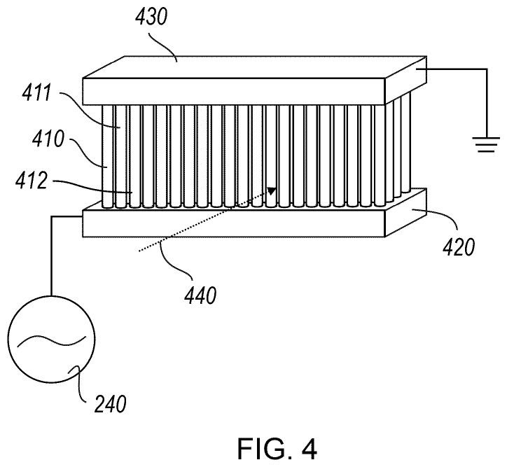

Referring to FIG. 4, the DBD element may include at least one electrode that comprises a plurality of small diameter high aspect ratio fibers 410, 411. This may include, e.g., a fabric such as velvet. A second electrode 430 may be present. The fibers may be coupled to a conductive plate 420. Each fiber may be identical, or some fibers may be different. In some embodiments, as seen in FIG. 3, at least one fiber 410 may include a conductive core with an outer non-conductive shell. In some embodiments, one or more fibers 411 may not include a conductive core. In some embodiments, one or more fibers 412 only include a conductive core. The DBD element may also include a second, grounded electrode 420. The fibers may be configured to allow a gas or aerosol to flow 440 through the plurality of fibers.

In some embodiments, a velvet material consisting of small diameter high aspect ratio fibers can be used as the part of the power electrode or ground electrode or both. FIG. 4 shows an example of the disclosed configuration with the power electrode having fiber velvet and the planar ground electrode made from a bulk material. The air passes through along the surface of the ground electrode and through the fibers. It is also possible to pass the air along the fibers normal to the ground electrode.

The gas or aerosol may be a liquid, or may include a liquid. The gas or aerosol may be a gas or may include a gas (such as air, and may include oxygen). The gas or aerosol may include an aerosol.

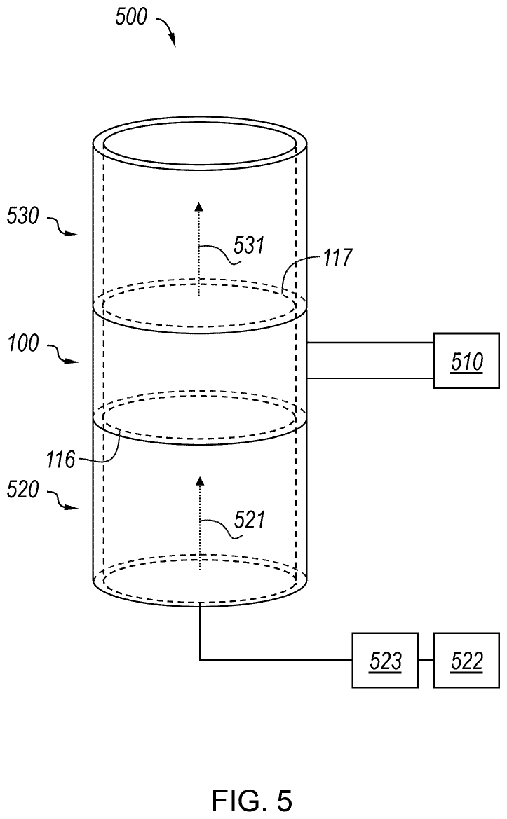

In various aspects, a system may be provided. The system may be a portable system. The system may be a non-portable system (e.g., it may be affixed to a roof, on a concrete pad on the ground, etc.) For example, referring to FIG. 5, the system 500 may include a device 100 as disclosed herein.

The system may include a power supply 510 (such as a battery) operably coupled to the device. The power supply may provide an AC current.

The system may include an inlet channel 520 configured to be operably connected to the inlet 116 and provide a path 521 directing at least one source 522 of a gas or aerosol to the device.

In some embodiments, the gas or aerosol may be a liquid. The system may include a nebulizer or atomizer 523 configured to utilize the source of the liquid to generate an aerosol, the aerosol being provided to the inlet channel.

The system may include an outlet channel 530 configured to be operably connected to the outlet 117 and provide a path 531 for a plasma-activated gas or aerosol to be transported away from the device.

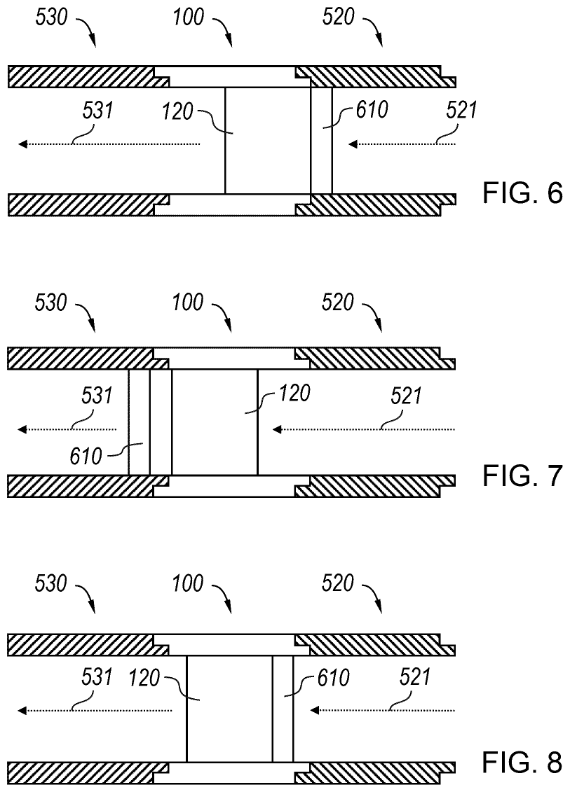

Referring to FIGS. 6-10, the system may include a gas or aerosol filter 610. The gas or aerosol filter may be disposed within the inlet channel (see FIG. 6). The gas or aerosol filter may be disposed within the outlet channel (see FIG. 7). The gas or aerosol filter may be disposed within the opening on the device (see FIGS. 8-10).

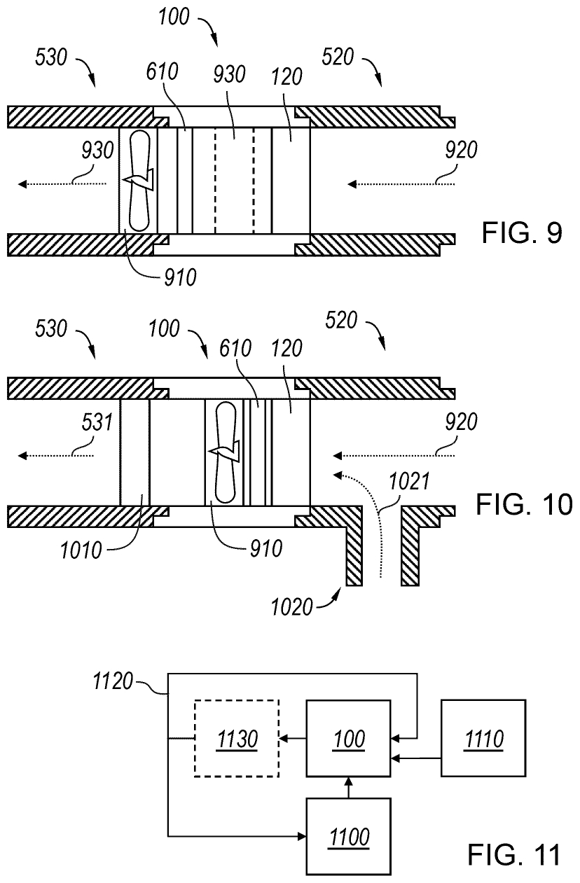

The system may include a fan 910. The fan may be positioned in the outlet channel (see FIG. 9). The fan may be positioned in the opening of the device (see FIG. 10).

The system may include a heating and/or cooling element 1010. The heating and/or cooling element may be positioned, e.g., in the outlet channel (see FIG. 10), in the opening of the device, or in the inlet channel. The heating and/or cooling element may include one or more electric heating coils (for heating). The heating and/or cooling element may include a Peltier device (for heating and/or cooling). The heating and/or cooling element may include one or more plates and/or tubes containing a heat-exchange fluid (for heating and/or cooling), such as a refrigerant, a glycol, etc.

Referring to FIGS. 9 and 10, the device may be configured to intake air from different locations. Referring to FIG. 9, in some embodiments, the inlet channel may be configured to receive air from a single source (e.g., to receive return air 920 from within an indoor space). The outlet chamber may be configured exhaust the air to a single location (e.g., to return air 930 to the indoor space it received air from). Referring to FIG. 10, the inlet channel may be configured to receive air from multiple sources (e.g., to receive return air 920 from within an indoor space and receive fresh air 1021 from an outdoor space). The inlet channel may include one or more ports 1020 configured to be coupled to each source of a gas (here, air).

In some embodiments, a recycling loop is included. For example, referring to FIG. 11, in some embodiments, a device may have multiple inputs and multiple outputs. The device may receive air from a room 1100 and from external air (e.g., from a location 1110 outside the building in which the room is located). Further, the device may receive air from a recirculation loop 1120.

Further, as seen in FIG. 11, one or more additional devices 1130 may (optionally) be operably coupled in series. The devices may have the same, or different, designs. For example, the first device (here, device 100) may have a flexDBD element and no filter, the second device (here, additional device 1130) may have weave DBD element and a filter.

The output from the device(s) may be split, part returning to the room 1100, and part entering the recycling loop 1120. The amount of gas or aerosol flowing through the recycling loop can be controlled using any known technique.

### Example 1

To make an embodiment of a disclosed device (here, a flexible printed circuit design, sometimes referred to as a “flexDBD” design), a printed-circuit-board technology was applied to manufacture a thin dielectric layer (e.g., polyimide, 100 microns thin), sandwiched between two thin layers of metal (e.g., copper ˜30 microns thin). The size of the sheet could vary. One of the metallic layers is meshed (typically square or round openings with dimensions of ˜1 mm and ˜1 mm gaps between them).

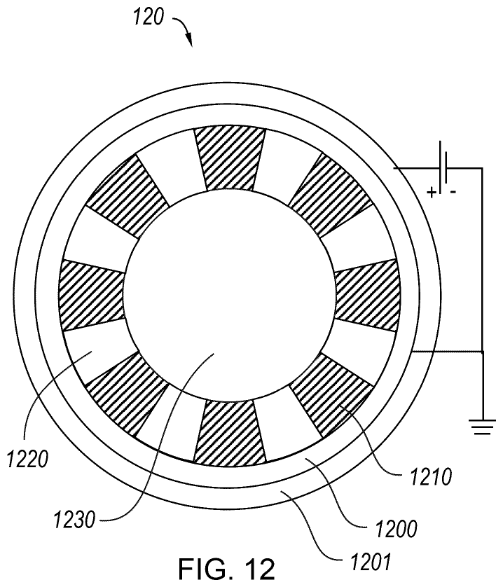

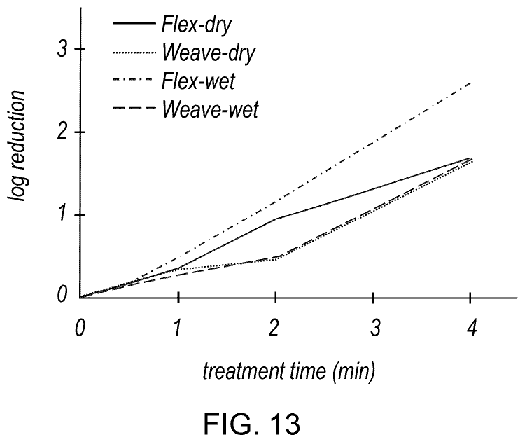

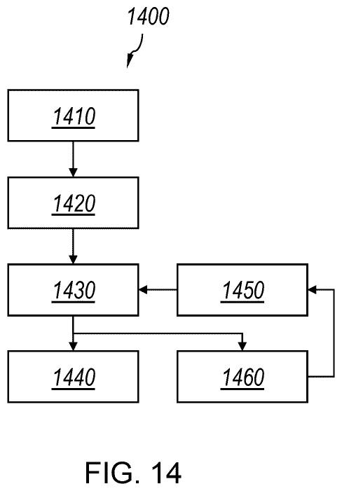

Referring to FIG. 12, the meshed layer 1200 was electrically grounded and the other metallic layer 1201 was connected to an AC power source (typically with 1-5 kV amplitude, 6-60 KHz frequency). Turning the power source “on” results in plasma 1210 generation in the holes of the mesh (here, 1220 are the structural parts of the meshed layer, with spaces between them representing the mesh).

FlexDBD can generally operate in ambient air, at atmospheric pressure, and room temperature without any additional gas flow at relatively low power densities (e.g., up to 0.5 W/cm²)

When the flexDBD sheet is in the tubular configuration, the meshed layer is toward the central axis, so the plasma is generated on the surface, inside the tube. This device can then be added as an extension to a nebulizer tube. Additional tubing (e.g., an outlet channel) to a mouthpiece or a mask can be added afterwards.

In addition to the use of a surface flex-DBD, a plasma fabric can be used to line the inside of the tube. Plasma fabric is a loom-woven fabric that uses two long fibers made from a soft insulating polymer material with a thin conductive core (see, e.g., FIG. 3). Each fiber is fully insulated and can be powered by a battery-operated power source. The power source may be built into the body of the nebulizer, or the body of the device. The size of the fabric can be changed as needed and requires only one electrical connection. This introduces additional options, as the fabric can be placed inside the tube, similar to that shown in FIG. 11, or over the opening (e.g., over opening 116) so that the mist passes through the fabric as it exits the nebulizer. Thus, in some embodiments, the DBD element may be coupled to an end of the device.

### Example 2

Referring to FIG. 12, two different embodiments of the devices (“flex” and “weave” in FIG. 12) were used to decontaminate gas or aerosol containing human herpes simplex virus (HSV), both as dried cultures (“dry”) and liquid cultures (“wet”). As seen, with the dried cultures, there was up to a 99% reduction in virus concentration, and up to a 99.7% reduction in the liquid cultures. Similar results were found with decontaminating SARS-COV2, where reductions of 90-95% for both devices were seen.

### Example 3

The various embodiments operate by creating disinfectant compounds from the gas or aerosols passing through the plasma. For example, plasma-activated mist has been tested for chemical reactivity by measuring the concentration of hydrogen-peroxide (H₂O₂) generated in a plasma-activated mist (for example, via the peroxone process) using, e.g., water testing strips. Hydrogen-peroxide is usually associated with plasma-induced reactivity and beneficial germicidal effects. The addition of the reactive nitrogen species may increase the disinfection efficiency of the hydroxyl radicals and other oxygen species.

This test has shown that exposing the mist to the plasma (including the devices in Example 1) introduces ˜10 ppm (mg/L) of H₂O₂. Flex and weave devices of similar sizes produce similar concentrations of reactive species.

In the current form, the disclosed device can be easily integrated into commercial medical nebulizers. It can be distributed as an add-on to the nebulizer and will only require very simple tube adapters for integration. The device can be adopted to larger scale mist generators used for example, in agriculture. The disclosed device can also be used for quick disinfection of large and complex surfaces, for example—cleaning of leafy vegetables at home or in agriculture.

In the disclosed device, rather than disinfecting surfaces, the chemical reactivity is delegated to the little liquid droplets that has very large potential to disperse and reach small crevices and therefore disinfect otherwise unreachable portions of complex surfaces with intricate topology.

A flexible or malleable DBD device produces uniform, cold plasma which can be applied for sterilization and personal hygiene purposes. The device can easily be made into a desirable geometry, in order to mount on a curved surface, such as door handle, for example. Cold plasma of DBD discharge has been previously shown to have beneficial biological effects and promote significant killing of bacteria and virus inactivation, without inducing thermal or other damage.

The effect of plasma from the flexible DBD can be enhanced with various liquids, enhancing the plasma-induced chemical reactivity. The device is very simple in operation, constructed from inexpensive components and powered by a simple and compact power source. Cold plasma devices produce cold plasma that is capable of decontaminating air flow from viruses and bacteria.

Disclosed herein are several plasma source configurations that can be easily incorporated into air-flow ways in buildings that are routinely occupied by people, such as residential housing and workplaces. This includes an application for some existing plasma disinfection devices and a development of a device. The disclosed approach includes several devices and several different configurations of existing devices. Existing plasma devices are not intended for industrial-scale decontamination.

The disclosed approach may be employed for decontamination of gas (particularly air) from bacterial, viral, and some chemical contaminants, and may be used in air ventilation systems with heating and/or air conditioning with re-circulation in residential and commercial buildings including hospitals. It also may be used as a part of a portable system for air decontamination in rooms including hospital rooms.

The disclosed approach is based on a multi-discharge plasma source. The disclosed multi-discharge system may include, but is not limited to multi-cavity, or multi-gap scalable and flexible systems including in some configurations field enhancement in the discharge gap around small diameter fiber electrodes. These systems may use dielectric barriers to limit the discharge current.

The disclosed design variations may include a use of, e.g., existing flexible dielectric barrier plasma source (flexDBD), configurations of the woven fiber design (plasma weave), and a dielectric barrier configuration utilizing a field enhancing fiber bristles such as in velvet materials.

The advantages of these designs include the ability to adopt to a variety of shapes such as, for example, the corrugated shape of air filters (flex-DBD and plasma weave), the ability to reduce the ignition voltage of the discharge (plasma velvet), scalability, safety, and portability.

The disclosed devices can be used as independent stand-alone or portable units as well as integrated into the existing air circulation systems, such as ventilation, heating, and air conditioning systems.

Several examples of disclosed configurations for air decontamination using the flex-DBD, plasma weave, and plasma velvet, are described below. In all configurations, the treated air can pass through the plasma device as many times as needed to achieve the desired level of disinfection. The air that is considered contaminated by viruses or bacteria-containing aerosols or airborne particles) passes through the plasma-based decontamination device and is pumped back into the occupied spaces.

Referring to FIG. 9, it can also be seen that in some embodiments, devices may optionally include one or more additional DBD elements 930 in the device, preferably in series with DBD element 120. The various DBD elements may be identical. The various DBD elements may be different.

A flexible DBD device is well-suited for such applications where large area is to be treated for a long duration. Such a device is capable of generating cold, homogeneous plasma in ambient atmosphere. The FlexDBD is readily made with printed circuit board (PCB) technology, and its effectiveness as an antimicrobial treatment in wound healing has previously been demonstrated. The device is also safe to touch due to very low current <1mA to the user and temperature T that is slightly above room temperature (22° C.<T <40° C.).

Plasma weave is easily scalable to large areas, flexible, and can be easily incorporated into the existing air filtering systems and used in any geometrical shape. For example, it can wrap the inner surfaces of the ductwork to prevent adsorption of the contaminants to the surface of the air ducts. Plasma weave can operate as a one-barrier or two barrier discharge with one or two insulated wires.

A device based on an array of high aspect ratio small diameter fibers may be geometrically positioned to reduce the discharge ignition voltage. The fiber materials for this device are available with conductive and non-conducting properties and in combinations of conducting and non-conducting fibers.

In various aspects, a method for air and aerosol decontamination may be provided. The method 1400 may include providing 1410 a device as disclosed herein. The method may include generating 1420 a plasma by causing a current to pass across the device. The method may include allowing 1430 a gas or an aerosol to pass into an inlet of the device, through the plasma, and out of an outlet.

In some embodiments, the method may include conducting 1440 the gas or aerosol to a location in a building or to a mask. In some embodiments, the method may include recycling 1450 at least a portion of the gas or aerosol passing out of the outlet to the inlet.

In some embodiments, the method may include controlling 1460 how much gas or aerosol is recycled, e.g., based on a measured flow rate of the gas or aerosol. This may allow the system to control the length of average length of time the gas or aerosol is exposed to the plasma, thereby controlling the efficacy of the system.

In various aspects, a kit may be provided. The kit may include a device as disclosed herein. The kit may include a filter. The kit may include a fan. The kit may include a heating and/or cooling element. Alternatively, the kit may include a device as disclosed herein, a liquid source or reservoir for liquid; a nebulizer or atomizer, and a power supply.

Various modifications may be made to the systems, methods, apparatus, mechanisms, techniques and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

What is claimed is:

1. A device comprising:
a housing having walls defining an opening therethrough, the opening configured to allow a gas or aerosol to pass from an inlet, through the housing, to an outlet; and
a dielectric barrier discharge (DBD) element positioned in the opening or covering an end of the opening, the DBD element configured to generate a plasma and configured to allow the gas or aerosol to pass along or through a surface of the DBD element.

2. The device according to claim 1, wherein the DBD element comprises a conductive plate and a grounded metal mesh, separated by a thin dielectric film.

3. The device according to claim 1, wherein the DBD element comprises a plurality of conductive wires configured in a mesh or mesh-like pattern.

4. The device according to claim 1, wherein the DBD element comprises a plurality of electrodes, wherein at least one electrode comprises a plurality of small diameter high aspect ratio fibers.

5. The device according to claim 1, wherein the gas or aerosol is a gas.

6. The device according to claim 1, wherein the gas or aerosol comprises an aerosol.

7. The device according to claim 1, wherein the DBD element is coupled to at least a portion of an inner surface of the walls defining the opening therethrough.

8. A system comprising:
a device according to claim 1;
a power supply operably coupled to the device;
an inlet channel configured to be operably connected to the inlet and provide a path directing at least one source of a gas or aerosol to the device; and
an outlet channel configured to be operably connected to the outlet and provide a path for a plasma-activated gas or aerosol to be transported away from the device.

9. The system according to claim 8, further comprising a gas or aerosol filter positioned in the outlet channel or inlet channel.

10. The system according to claim 8, further comprising a fan positioned in the outlet channel.

11. The system according to claim 8, further comprising a heating and/or cooling element positioned in the outlet channel.

12. The system according to claim 8, wherein the inlet channel is configured to receive return air from within an indoor space and the outlet channel is configured to return air to the indoor space.

13. The system according to claim 12, wherein the inlet channel is configured to receive return air from within an indoor space and fresh air from an outdoor space.

14. The system according to claim 8, wherein the inlet channel is coupled to a source of a liquid.

15. The system according to claim 14, further comprising a nebulizer or atomizer configured to utilize the source of the liquid to generate an aerosol.

16. The system according to claim 8, wherein the power source is a battery.

17. The system according to claim 8, wherein the system is portable.

18. A method for air and aerosol decontamination, comprising:
providing a device according to claim 1;
generating a plasma by causing a current to pass across the device; and
allowing a gas or an aerosol to pass into an inlet of the device, through the plasma, and out of an outlet.

19. A kit comprising:
a device according to claim 1;
a filter; and
a fan.

20. The kit according to claim 19, further comprising a heating and/or cooling element.

21. A kit comprising:
a device according to claim 1;
a liquid source or reservoir for liquid; a nebulizer or atomizer; and
a power supply.
